(19) United States
(12) Patent Application Publication
(10) Pub. No.: US 2016/0251216 A1
(43) Pub. Date: Sep. 1, 2016

(54) PHOTOCHEMICAL REACTION DEVICE, METHOD FOR MANUFACTURING SAME, AND PHOTOCHEMICAL REACTION METHOD

(71) Applicants: NAGOYA INSTITUTE OF TECHNOLOGY, Aichi (JP); NATIONAL INSTITUTE OF ADVANCED INDUSTRIAL SCIENCE AND TECHNOLOGY, Chiyoda-ku, Tokyo, OT (JP)

(72) Inventors: Masaharu KONDO, Nagoya-city, Aichi (JP); Tomoyasu NOJI, Osaka-city, Osaka (JP); Takehisa DEWA, Nagoya-city, Aichi (JP); Tetsuro JIN, Ikeda-city, Osaka (JP)

(73) Assignee: NATIONAL INSTITUTE OF ADVANCED INDUSTRIAL SCIENCE AND TECHNOLOGY, Chiyoda-ku, Tokyo, OT (JP)

(21) Appl. No.: 14/909,601

(22) PCT Filed: Jul. 30, 2014
(86) PCT No.: PCT/JP2014/070102

§ 371 Date: May 12, 2016

(30) Foreign Application Priority Data
Aug. 2, 2013 (JP) 2013-161622

(51) Int. Cl. C01B 3/02; B01J 19/12

(52) U.S. Cl. CPC C01B 3/02; B01J 19/127; B01J 2219/1203

(57) ABSTRACT

To provide a photochemical reactor that allows a photochemical reaction to proceed in the atmospheric air. Used is porous glass that is permeable to light in such a wavelength region as to be absorbable by photosensitizer. The porous glass has pores and includes the photosensitizer, electron carrier, and reduction reaction catalyst, each serving as a reaction-involved substance and being disposed in the pores. This allows the interior of the pores of the porous glass to serve as a reaction site for the photochemical reaction and allows the photochemical reaction to proceed in the atmospheric air.

### TECHNICAL FIELD

The present invention relates to a photochemical reactor that promotes a chemical reaction using energy of light such as sunlight, to a method for producing the photochemical reactor, and to a photochemical reaction method using the photochemical reactor.

### BACKGROUND ART

FIGS. 5(a) and 5(b) illustrate a light-induced hydrogen evolution reaction. As illustrated in FIGS. 5(a) and 5(b), photosensitizer (P) is photoexcited via light irradiation to pass an electron (e⁻) to electron carrier (C). The electron carrier (C) further passes the electron (e⁻) to a reduction reaction catalyst (Catalyst), and this allows hydrogen (H₂) to evolve from hydrogen ions (H⁺). The photosensitizer (P), which has lost the electron (e⁻), receives another electron (e⁻) from an electron donor (D).

Assume that oxygen molecules are present in a large amount in the reaction system in the light-induced hydrogen evolution reaction. In this case, most of electrons flow from the electron carrier (C) to the oxygen molecules (O₂), and this impedes the evolution of hydrogen by the action of the catalyst (Catalyst), as illustrated in FIG. 5(b). In general, oxygen has to be removed from the reaction system so as to promote the light-induced hydrogen evolution reaction. This is also true for not only the light-induced hydrogen evolution reaction, but also other photochemical reactions by the medium of electron carrier that is readily oxidized by an oxygen molecule.

In contrast, Patent Literature (PTL) 1 and PTL 2 describe that an improved compound derived from methylviologen, when used as electron carrier, allows a photochemical reaction to proceed in the atmospheric air even when oxygen is not removed from the reaction system. The improved compound derived from methylviologen is a compound that corresponds to methylviologen, except for further containing an alkylene group so as to constitute a structure that can form a micelle in an aqueous medium. The improved compound derived from methylviologen forms a micelle in an aqueous medium, and whose reductant has a longer lifetime even in the presence of dissolved oxygen in the aqueous medium, as compared with methylviologen. This allows the photochemical reaction to proceed in the atmospheric air.

PTL 1 (see paragraph [0057]) describes that methylviologen is not reduced when used in the atmospheric air.

### CITATION LIST

### Patent Literature

PTL 1: Japanese Patent Application Publication No. JP-A-2008-237160

PTL 2: Japanese Patent Application Publication No. JP-A-2010-63453

### SUMMARY OF INVENTION

### Technical Problem

The conventional technologies allow the photochemical reaction to proceed in the atmospheric air not by improving the reaction site, but by using, as electron carrier, the improved compound derived from methylviologen.

Under these circumstances, the present invention has an object to provide a photochemical reactor that allows a photochemical reaction to proceed in the atmospheric air by improving the reaction site, and to provide a method for performing such a photochemical reaction using the photochemical reactor. The present invention has another object to provide a method for producing the photochemical reactor.

### Solution to Problem

To achieve the objects, a photochemical reactor according to a first aspect of the present invention includes a porous glass (1) having a lot of pores (2) and being permeable to light. The photochemical reactor also includes photosensitizer, electron carrier, and reduction reaction catalyst which are disposed in the pores (2).

Assume that a large amount of oxygen is present in the reaction site for a photochemical reaction by the medium of electron carrier that is readily oxidized by oxygen molecules. In this case, most of electrons flow from the electron carrier to oxygen. This restrains the electron carrier from passing the electrons to the catalyst and impedes the progress of the photochemical reaction, as described above.

In contrast, the photochemical reactor according to the present invention uses the interior of the pores in the porous glass as a reaction site for the photochemical reaction. The interior of pores in the porous glass resists oxygen migration thereinto. This decreases the amount of oxygen present in the reaction site even when the porous glass is disposed in the atmospheric air. This configuration decreases electrons that move from the electron carrier to oxygen, but increases electrons that move from the electron carrier to the catalyst, as compared with the case where a large amount of oxygen is present in the reaction site. The photochemical reactor according to the present invention, as having the configuration, allows the photochemical reaction to proceed in the atmospheric air.

In a second aspect of the photochemical reactor according to the first aspect, the porous glass may have an average pore diameter of 20 to 75 nm. The pores preferably have sizes within the above range.

In a third aspect of the photochemical reactor according to one of first and second aspects, the electron carrier may include at least one selected from the group consisting of methylviologen, cytochromes, methylene blue, and titanium oxides.

Methylviologen is not reduced in the atmospheric air, as described in PTL 1, and fails to allow the photochemical reaction to proceed in the atmospheric air, because methylviologen does not form a micelle in an aqueous medium.

In contrast, in the third aspect, the photochemical reactor according to the first or second aspect allows the photochemical reaction to proceed in the atmospheric air even when methylviologen is used as in the embodiment. As described above, the present invention offers advantageous effects that are not given by the conventional technologies.

According to a fourth aspect of the present invention, a method produces the photochemical reactor according to the first aspect. The method includes step for preparing a porous glass, and solution containing photosensitizer, electron carrier, and catalyst, and step for immersing the porous glass in the solution.

The photochemical reactor according to the first to third aspects can be produced in the above manner.

According to a fifth aspect of the present invention, a method performs a photochemical reaction using the photochemical reactor according to the first to third aspects. In the method, solution containing is supplied to the interior of the pores (2) in the porous glass (1), and light is applied to the porous glass (1). These are performed in the atmospheric air.

This method, as using the photochemical reactor according to the first to third aspects, allows the photochemical reaction to proceed in the atmospheric air, for reasons similar to those in the first aspect of the present invention.

Reference signs in the parentheses indicating the means (components) described herein and in the claims are examples illustrating correspondence relations with specific means (components) described in after-mentioned embodiments.

### BRIEF DESCRIPTION OF DRAWINGS

FIG. 1 is a conceptual diagram illustrating a photochemical reactor according to the present invention.

FIG. 2 illustrates a production process for the porous glass as illustrated in FIG. 1.

FIG. 3 illustrates a light-induced hydrogen evolution reaction performed in the atmospheric air using the photochemical reactor according to the present invention.

FIG. 4 is a graph illustrating the detected amount of hydrogen evolution in Example 1 according to the present invention and in Comparative Example 1.

FIG. 5 illustrates a light-induced hydrogen evolution reaction performed according to the conventional technologies.

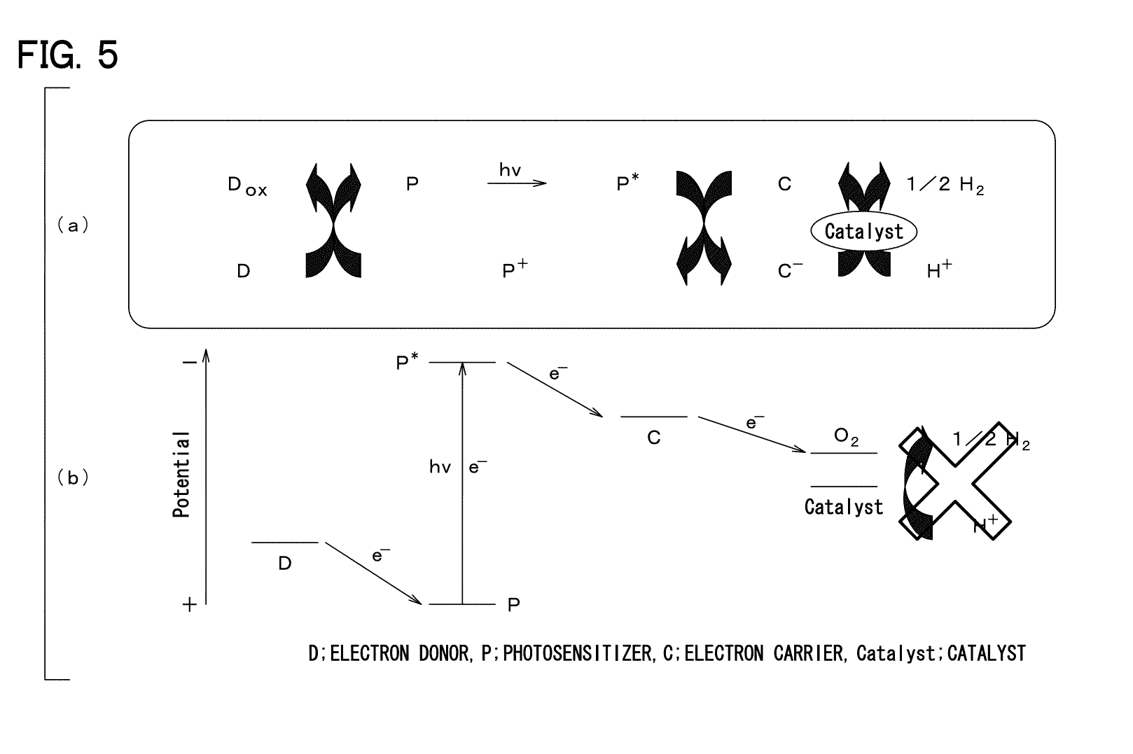

### DESCRIPTION OF EMBODIMENTS

Embodiments of the present invention will be described with reference to the attached drawings.

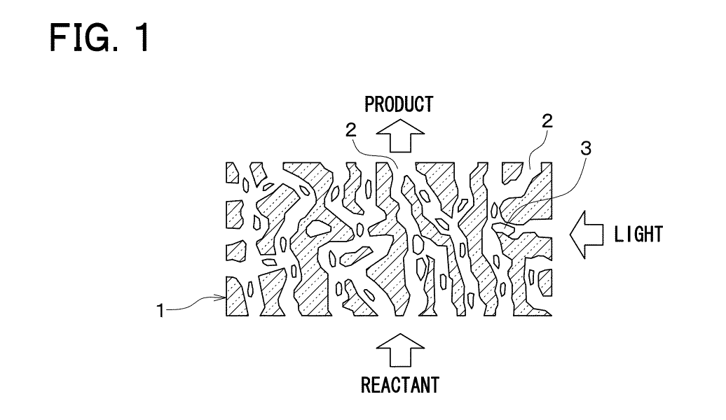

FIG. 1 is a conceptual diagram illustrating the photochemical reactor according to the present invention. As illustrated in FIG. 1, the photochemical reactor according to the present invention includes a porous glass 1. The porous glass 1 has a lot of pores 2 and is permeable to light. The photochemical reactor further includes reaction-involved substances 3 in the pores 2 of the porous glass 1. The reaction-involved substances 3 are involved in a photochemical reaction.

The reaction-involved substances 3 include an electron donor, photosensitizer, electron carrier, and reduction reaction catalyst. Examples of the photochemical reaction include, but are not limited to, a reaction by which hydrogen ions give hydrogen, where the hydrogen ions are reactant material, and hydrogen is product material; a reaction by which carbon dioxide gives formic acid; a reaction by which formaldehyde gives methanol; and a reaction by which oxaloacetic acid gives malic acid.

The pores 2 of the porous glass 1 are continuous and penetrate the porous glass 1. The pores 2 have only to have such sizes as to house the reaction-involved substances 3. More specifically, the porous glass 1 preferably has an average pore diameter (average pore size) of 20 to 75 nm (from 20 nm to 75 nm). The inventors of the present invention experimentally determined that the porous glass 1, when having an average pore diameter within the range, promotes the photochemical reaction in the atmospheric air. The pores preferably have a narrow range of variation in pore diameter. For example, pores distributed in a region at a pore diameter of within ±10% of the average pore diameter preferably occupy 90% or more of the total pore size distribution. The average pore diameter and pore size distribution may be measured by mercury porosimetry.

The porous glass 1 preferably has a sheet-like shape. The porous glass 1 may have such a thickness as to allow the light to penetrate the entire porous glass 1.

The light to penetrate the porous glass 1 may be one in the visible light region, but may also be one in another region, as long as being absorbable by the photosensitizer. In short, the porous glass 1 is selected from ones that are permeable to such light as to be absorbable by the photosensitizer.

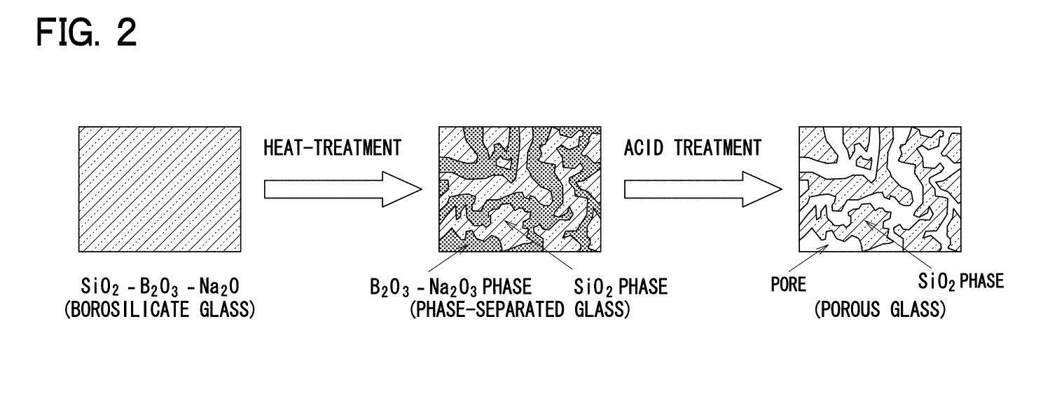

The porous glass 1 can be selected from regular ones, which are prepared using a glass phase splitting phenomenon. FIG. 2 illustrates a process for producing the porous glass 1, using the glass phase splitting phenomenon. Typically, the porous glass 1 may be prepared by the process illustrated in FIG. 2. In the process, a borosilicate glass having a composition including SiO₂, B₂O₃, and Na₂O is heat-treated to undergo spinodal phase splitting and further subjected to an acid treatment to melt the B₂O₃—Na₂O phase.

The porous glass 1 may be selected from not only ones produced by the melt process, but also ones produced by a sol-gel process. However, the porous glass 1 is preferably selected from ones produced by the melt process. This is because the porous glass produced by the melt process has higher mechanical strength, superior formability, and is more suitably configured into a device, as compared with the porous glass produced by the sol-gel process.

The electron donor has a reducing function, namely, the function of passing an electron to another substance. Upon the reduction, the electron donor itself is oxidized. Specifically, the “electron donor” refers to a substance that has the reduction function, namely, the function of passing an electron to the photosensitizer in the reaction system, where the photosensitizer has lost an electron upon light irradiation. The electron donor is also called a reductive sacrificial reagent. As used in this reaction system, the term “electron donor” refers to a substance that passes an electron to the photosensitizer, which has passed an electron to the electron carrier. Examples of the electron donor include, but are not limited to, ethylenediaminetetraacetic acid (EDTA), sodium ascorbate, triethanolamine, mercaptoethanol, nicotinamide adenine dinucleotide, and nicotinamide adenine dinucleotide phosphate.

The photosensitizer has such a role as to absorb light energy, to pass the light energy to another substance, and to assist the reaction and a light-emitting process. Specifically, the photosensitizer has a photoelectric conversion function in which the photosensitizer absorbs light energy, transforms the light energy to the energy of electron, and passes the electron to the electron carrier, in the light-irradiated reaction system. Examples of the photosensitizer usable herein include metallic compounds and organic compounds, such as ruthenium metal complexes, porphyrin derivatives, phthalocyanine derivatives, chlorophyll derivatives; and protein-chlorophyll pigment complexes, which are extracted from photosynthetic organisms. The extraction from photosynthetic organisms requires surfactant n-dodecyl maltoside so as to disperse the extracted substance uniformly in an aqueous solution.

The electron carrier has an electron transport function, in which the electron carrier receives an electron and passes the electron to another substance. Specifically, the electron carrier has a reducing function, in which the electron carrier receives an electron from the photosensitizer, which has been photoexcited by light irradiation, and passes the electron to the catalyst. The electron carrier is also called an electron transporter or an electron medium. Examples of the electron carrier include, but are not limited to, methylviologen, quinone derivatives, indophenol, titanium oxides, methylene blue, Janus green B, and cytochromes.

The reduction reaction catalyst is a substance that increases the reaction rate of a specific chemical reaction, but does not change itself between before and after the reaction. In the light-irradiated reaction system, the catalyst receives the electron from the electron carrier and reduces reactant material into product material. Non-limiting examples of the catalyst include metal catalysts such as platinum catalysts; and enzymes. Examples of the enzymes include, but are not limited to, hydrogenases, alcohol dehydrogenases, formate dehydrogenases, and malic acid dehydrogenases. The catalyst may be selected according to the type of the photochemical reaction. For example, the catalyst may be selected from hydrogenases and platinum for the hydrogen evolution reaction; selected from formate dehydrogenases for the formic acid forming reaction; selected from alcohol dehydrogenases for the methanol forming reaction; and selected from malic acid dehydrogenases for the malic acid forming reaction.

These reaction-involved substances 3 are adsorbed by, and immobilized on, the surface of the pores 2 of the porous glass 1. The porous glass 1 including the reaction-involved substances 3 immobilized on the surface of the pores 2 may be produced in the following manner.

Initially, a first step is performed by which the porous glass 1, and solution containing the electron donor, the photosensitizer, the electron carrier, and the catalyst in a solvent are prepared. Non-limiting examples of the solvent include aqueous solvents. Such aqueous solvents may have buffering actions.

Next, a second step is performed by which the porous glass 1 is immersed in the solution. This gives the porous glass 1 that includes the reaction-involved substances 3 adsorbed by, and immobilized on, the surface of the pores 2. To firmly immobilize the reaction-involved substances 3 on the surface of the pores 2, the porous glass 1 may be subjected to a modification treatment on the surface of the pores 2 before the immersion so as to immobilize the reaction-involved substances 3 onto the surface of the pores 2 typically via chemical bonding.

Upon the photochemical reaction, the porous glass 1 after the immersion may be used without drying. Alternatively, the porous glass 1 after the immersion may be washed, and the porous glass 1 after the washing may be used without drying. The absence of drying is for the purpose of eliminating or minimizing fracture of an enzyme, when the enzyme is used as the catalyst. Unless the catalyst undergoes fracture, the porous glass 1 after the immersion or washing may be dried before use.

The porous glass 1 is irradiated with light upon use of the photochemical reactor. Non-limiting examples of a light source to irradiate the porous glass 1 with light includes sun and simulated sunlight generators.

Next, a method for performing a photochemical reaction using the photochemical reactor having the configuration will be described.

As illustrated in FIG. 1, solution containing reactant material in a solvent is supplied into the pores 2 of the porous glass 1. Specifically, the porous glass 1 is immersed in the solution containing the reactant material to supply the solution containing the reactant material into the pores 2. In this state, the solution containing the reactant material is pooled in a reaction vessel. Non-limiting examples of the solvent include aqueous solvents. The aqueous solvents may have buffering actions. The porous glass 1 is then irradiated with light. This allows the photochemical reaction to proceed and to reduce the reactant material into the product material, without an operation of removing oxygen from the reaction system.

The solution containing the reactant material before the immersion of the porous glass 1 preferably further contains an electron donor. This allows not only the electron donor previously immobilized in the interior of the pores, but also the electron donor in the solution, to impart electrons to the photosensitizer. This allows the photochemical reaction to give the product material in a larger amount.

In addition, the photochemical reaction can be continued by adding another portion of the electron donor to the solution containing the reactant material after the photochemical reaction.

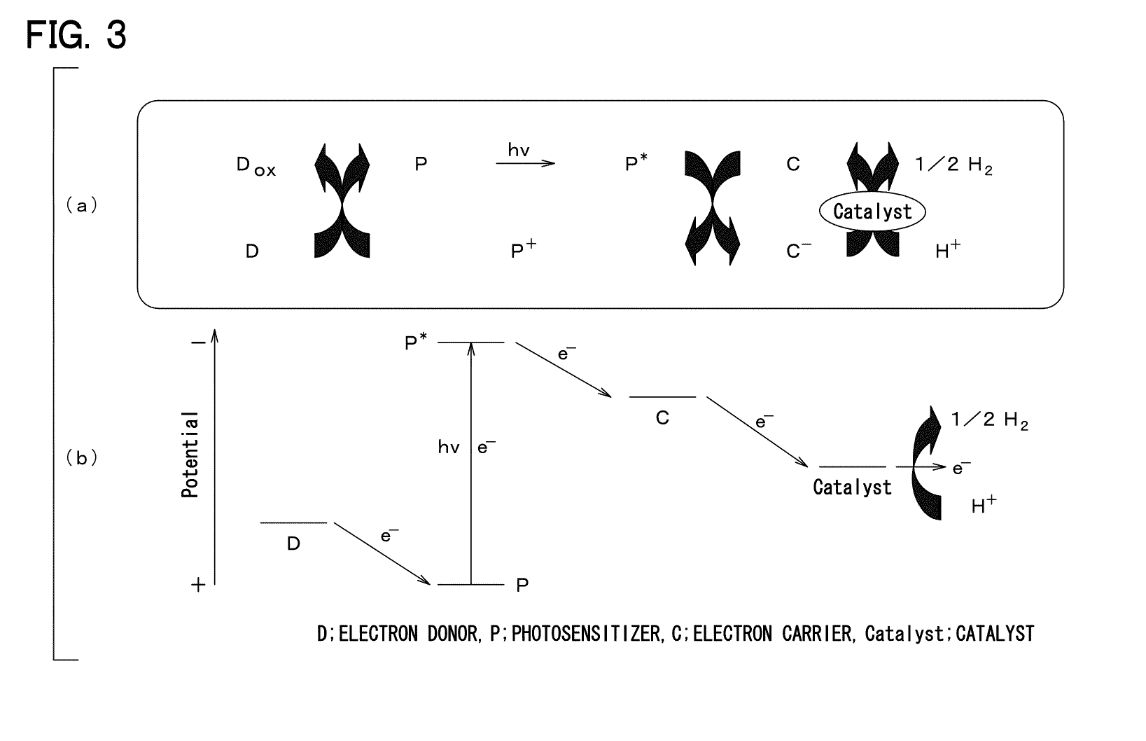

FIGS. 3(a) and 3(b) illustrate a light-induced hydrogen evolution reaction using the photochemical reactor according to the present invention. As illustrated in FIGS. 3(a) and 3(b), the photochemical reactor according to the present invention upon use allows the light-induced hydrogen evolution reaction to proceed in the atmospheric air. This is because as follows.

The photochemical reactor having the configuration employs the porous glass 1 that is permeable to light in such a region as to be absorbable by the photosensitizer. The photochemical reactor includes the reaction-involved substances 3 disposed in the pores 2. This allows the interior of the pores 2 in the porous glass 1 to act as a reaction site for the photochemical reaction. The interior of the pores 2 in the porous glass 1 resists migration of oxygen thereinto. This decreases the amount of oxygen present in the reaction site even when the porous glass 1 is placed in the atmospheric air.

This decreases the amount of electrons that move from the electron carrier to oxygen, but increases the amount of electrons (e⁻) that move from the electron carrier (C) to the catalyst (Catalyst) as illustrated in FIG. 3(b), as compared with the case where a large amount of oxygen is present in the reaction site. Accordingly, the photochemical reactor according to the present invention allows the light-induced hydrogen evolution reaction to proceed in the atmospheric air. This is also true for any other photochemical reactions.

The photochemical reactor having the configuration may be produced by immersing the porous glass 1 in solution containing the reaction-involved substances 3. When the porous glass 1 is immersed in the solution containing the reaction-involved substances 3 to adsorb the reaction-involved substances 3 on the pores 2 surface, the reaction-involved substances 3 exist more densely on the pores 2 surface, as compared with the solution. The photochemical reactor having this configuration can constitute a high-concentration reaction site.

The above-described embodiments are not construed to limit the scope of the present invention, and various changes, variations, and modifications are possible as appropriate within the scope and sprit of the present invention as defined in the appended claims.

In the embodiments, the electron donor, the photosensitizer, the electron carrier, and the catalyst are previously disposed in the pores 2 of the porous glass 1 in the photochemical reactor. However, the electron donor may be disposed in the pores 2 afterward. For example, the electron donor may be supplied together with the reactant material into the pores 2 upon the photochemical reaction. In short, the electron donor has only to be disposed in the pores 2 at the time when the photochemical reaction is performed. This may be achieved not only by disposing the electron donor with other substances involved in the photochemical reaction in the pores 2 before the reaction, but also by supplying the electron donor with the reactant material into the pores 2 upon the photochemical reaction.

In a preferred embodiment, the electron donor is disposed in the pores 2 of the porous glass 1 before the reaction and is also supplied as in the solution containing the reactant material upon the photochemical reaction. This configuration speeds up the initiation of the photochemical reaction.

In the embodiments, the porous glass 1 is immersed in the solution containing the reactant material, which is pooled in the reaction vessel, upon the photochemical reaction using the photochemical reactor. In another embodiment, the porous glass 1 may be immersed in the solution containing the reactant material and flowing in a channel to continuously supply the solution into the pores 2.

In the embodiments, the solution containing the reaction-involved substances 3 and the solution containing the reactant material are separately prepared, the porous glass 1 is immersed in the solution containing the reaction-involved substances 3 to immobilize the reaction-involved substances 3 onto the pores 2 surface, and thereafter the porous glass 1 is immersed in the solution containing the reactant material. In another embodiment, the porous glass 1 may be immersed in solution containing both the reaction-involved substances 3 and the reactant material. Specifically, both the reaction-involved substances 3 and the reactant material may be supplied simultaneously into the pores 2 of the porous glass 1.

In the embodiments, the reaction-involved substances 3 are immobilized on the pores 2 surface in the porous glass 1. In another embodiment, the reaction-involved substances 3 may be not immobilized on the pores 2 surface, but merely disposed in the pores 2 of the porous glass 1.

In the embodiments, the electron carrier has such a reduction function as to receive an electron from the photosensitizer and to pass the electron to the catalyst. In another embodiment, the electron carrier may have such a function as to receive an electron from the electron donor and to pass the electron to the photosensitizer. In this embodiment, the photosensitizer has such a function as to pass an electron to the catalyst. Non-limiting examples of the electron carrier having this function include cytochromes (see Example 3).

### EXAMPLES

The present invention will be illustrated in further detail with reference to several examples below. Porous glass used in Examples 1, 2, and 3 was prepared by the melt process, had a sheet-like shape with a thickness of 1 mm, and had an average pore diameter of 50 nm. Porous glass used in Examples 4, 5, and 6 was prepared by the sol-gel process, had a particulate shape with a diameter of 3 to 5 mm, and had an average pore diameter of 50 nm. In both the porous glass used in Examples 1 to 3 and the porous glass used in Examples 4 to 6, pores distributed in a region at a pore diameter of 45 to 55 nm occupy 90% or more of the total pore size distribution (total pores). The pore diameter of the porous glass was measured using Automatic Porosimeter AutoPore IV 9500 (supplied by Shimadzu Corporation).

### Example 1

There was prepared solution containing 20 mM ethylenediaminetetraacetic acid (EDTA) as an electron donor; 0.5 mM ruthenium metal complex Ru(bpy)₃ as photosensitizer; 3.0 mM methylviologen as electron carrier; and 5.5 μM hydrogenase as catalyst, in a 100 mM Tris buffer (pH 8.0). The porous glass 1 (10 mg) was immersed in 133 μL of the solution for 12 hours or longer to adsorb the reaction-involved substances 3 on the pores 2 surface (see FIG. 1).

Next, the porous glass after immersion was retrieved from the solution and immersed once in 1.33 mL of a 100 mM Tris buffer (pH 7.4) containing 20 mM ethylenediaminetetraacetic acid (EDTA), to cleanse the porous glass surface.

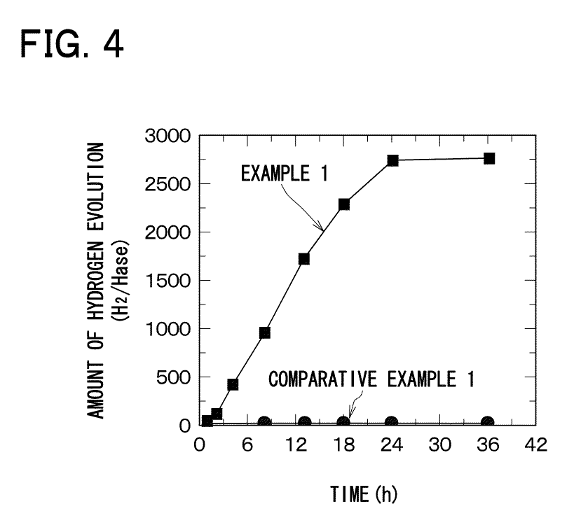

The porous glass 1 adsorbing the reaction-involved substances 3 was placed in 1.33 mL of another portion of the 100 mM Tris buffer (pH 7.4) containing 20 mM ethylenediaminetetraacetic acid (EDTA), where the buffer was contained in a glass vessel. The glass vessel was closed up with a rubber stopper and was used as a reaction vessel. The porous glass 1 was irradiated with light from a simulated sunlight generator to promote a photochemical reaction. Gases were collected from the glass vessel using a syringe, and evolved hydrogen gas was detected and measured by gas chromatography. The results are given in FIG. 4. The results in FIG. 4 demonstrate that a light-induced hydrogen evolution reaction proceeded in the atmospheric air. The hydrogen evolution amount (H₂/Hase) indicated on the ordinate of FIG. 4 is a hydrogen evolution amount per molecule of the hydrogenase.

### Comparative Example 1

Solution as in Example 1 used for the adsorption of the reaction-involved substances 3 by the porous glass 1 was prepared. An aliquot (150 μL) of the solution was placed in a glass vessel that can be covered with a rubber stopper as a lid, and the solution was irradiated with light from a simulated sunlight generator, to make an attempt to perform a photochemical reaction. Gases were collected from the glass vessel using a syringe, and evolved hydrogen gas was detected and measured by gas chromatography. The results are given in FIG. 4. The results in FIG. 4 demonstrate that no hydrogen gas was detected, and the photochemical reaction did not proceed.

### Example 2

There was prepared solution containing a protein-chlorophyll pigment complex extracted from a photosynthetic organism (light-harvesting protein, 24 μM) as photosensitizer; 3.0 mM methylviologen as electron carrier; 1.4 μM hydrogenase as catalyst; and 0.03% (w/v) n-dodecyl maltoside, in a 100 mM Tris buffer (pH 7.4). The porous glass 1 (10 mg) was immersed in 133 μL of the solution for 12 hours or longer to adsorb the reaction-involved substances 3 on the pores 2 surface (see FIG. 1).

Next, the porous glass after immersion was retrieved from the solution, and immersed once in 1.33 mL of a 100 mM Tris buffer (pH 7.4) containing 20 mM nicotinamide adenine dinucleotide, to cleanse the glass surface.

The porous glass 1 adsorbing the reaction-involved substances 3 was placed in 1.33 mL of a 100 mM Tris buffer (pH 7.4) containing 20 mM nicotinamide adenine dinucleotide as an electron donor, where the buffer was contained in a glass vessel. The glass vessel was closed up with a rubber stopper and was used as a reaction vessel. The porous glass 1 was irradiated with light from a simulated sunlight generator to promote a photochemical reaction. Gases were collected from the glass vessel using a syringe, and evolved hydrogen gas was detected and measured by gas chromatography. As a result, 1 nmol of hydrogen gas were detected after light irradiation for 7 hours, which is smaller as compared with Example 1. The result of this is not shown in the graph.

### Example 3

This example illustrates a photo-induced hydrogen evolution system. The system employed a protein-chlorophyll pigment complex (photochemical system I protein-pigment complex) extracted from a photosynthetic organism and serving as photosensitizer; cytochrome c6 as electron carrier; colloidal platinum as catalyst; and sodium ascorbate as an electron donor. In this example, cytochrome c6 (electron carrier) has such a function as to receive an electron from sodium ascorbate (electron donor) and to pass the electron to the protein-chlorophyll pigment complex (photo sensitizer).

Initially, the protein-chlorophyll pigment complex and the colloidal platinum were previously mixed in proportions in concentration of 1:2 in a 20 mM Tris buffer (pH 8.0) to give a complex between the protein-chlorophyll pigment complex and the colloidal platinum. Next, there was prepared solution containing the resulting complex (0.3 mg Chl/mL, chlorophyll weight concentration), 50 mM cytochrome c6, 100 mM sodium ascorbate, and 0.03% (w/v, weight per volume concentration) n-dodecyl maltoside in a 40 mM HEPES buffer (pH 7.8). The porous glass 1 (18.3 mg) was immersed in 244 mL of the solution for 12 hours or longer to adsorb the reaction-involved substances 3 on the pores 2 surface (see FIG. 1).

Next, the porous glass after immersion was retrieved from the solution, and immersed once in 2.44 mL of a 40 mM MES buffer (pH 6.2) containing 100 mM sodium ascorbate, to cleanse the glass surface.

The porous glass 1 adsorbing the reaction-involved substances 3 was placed in 2.44 mL of a 40 mM MES buffer (pH 6.0) containing 100 mM sodium ascorbate as an electron donor and 4 mM cytochrome c6 as electron carrier, where the buffer was contained in a glass vessel. The glass vessel was closed up with a rubber stopper and was used as a reaction vessel. The porous glass 1 was irradiated with light from a simulated sunlight generator to promote a photochemical reaction. Gases were collected from the glass vessel using a syringe, and evolved hydrogen gas was detected and measured by gas chromatography. As a result, 4.5 nmol of hydrogen gas were detected after light irradiation for 6.5 hours.

### Example 4

Particulate porous glass was prepared by the sol-gel process. The reaction-involved substances 3 as in Example 1 were adsorbed by the particulate porous glass on the surface of pores under conditions similar to Example 1.

Next, the particulate porous glass after immersion was retrieved from the solution, and immersed once in 1.33 mL of a 100 mM Tris buffer (pH 7.4) containing 20 mM ethylenediaminetetraacetic acid (EDTA), to cleanse the particulate porous glass surface.

The particulate porous glass adsorbing the reaction-involved substances 3 was placed in 1.33 mL of another portion of the 100 mM Tris buffer (pH 7.4) containing 20 mM ethylenediaminetetraacetic acid (EDTA), where the buffer was contained in a glass vessel. The glass vessel was closed up with a rubber stopper and was used as a reaction vessel. The particulate porous glass was irradiated with light from a simulated sunlight generator to promote a photochemical reaction. Gases were collected from the glass vessel using a syringe, and evolved hydrogen gas was detected and measured by gas chromatography. After light irradiation for 24 hours, 90 nmol of hydrogen gas were detected.

### Example 5

There was prepared solution containing 20 mM ethylenediaminetetraacetic acid (EDTA) as an electron donor; 0.5 mM ruthenium metal complex Ru(bpy)₃ as photosensitizer; 3.0 mM methylene blue as electron carrier; and 5.5 μM hydrogenase as catalyst, in a 100 mM Tris buffer (pH 8.0). Separately, a particulate porous glass was prepared by the sol-gel process, and 10 mg of the particulate porous glass 1 was immersed in 1.33 μL of the solution for 12 hours or longer to adsorb the reaction-involved substances 3 on the pores 2 surface (see FIG. 1).

Next, the porous glass after immersion was retrieved from the solution, and immersed once in 1.33 mL of a 100 mM Tris buffer (pH 7.4) containing 20 mM ethylenediaminetetraacetic acid (EDTA), to cleanse the porous glass surface.

The porous glass 1 adsorbing the reaction-involved substances 3 was placed in 1.33 mL of another portion of the 100 mM Tris buffer (pH 7.4) containing 20 mM ethylenediaminetetraacetic acid (EDTA), where the buffer was contained in a glass vessel. The glass vessel was closed up with a rubber stopper and was used as a reaction vessel. The porous glass 1 was irradiated with light from a simulated sunlight generator to promote a photochemical reaction. Gases were collected from the glass vessel using a syringe, and evolved hydrogen gas was detected and measured by gas chromatography. As a result, 5 nmol of hydrogen gas were detected after light irradiation for 24 hours.

### Example 6

The porous glass (4.9 mg) was immersed in 133 μL of a 1.0 mg/mL aqueous solution of titanium oxide (having a particle diameter of about 40 nm) for 12 hours or longer to adsorb titanium oxide as electron carrier. The porous glass was then washed with 1.33 mL of distilled water. Separately, there was prepared solution containing 20 mM ethylenediaminetetraacetic acid (EDTA) as an electron donor; 0.5 mM ruthenium metal complex Ru(bpy)₃ as photosensitizer; and 5.5 μM hydrogenase as catalyst, in a 100 mM Tris buffer (pH 8.0). The porous glass after washing was immersed in 133 μL of the solution for 12 hours or longer to adsorb all the reaction-involved substances 3 on the pores 2 surface (see FIG. 1).

Next, the porous glass after immersion was retrieved from the solution, and immersed once in 1.33 mL of a 100 mM Tris buffer (pH 7.4) containing 20 mM ethylenediaminetetraacetic acid (EDTA), to cleanse the porous glass surface.

The porous glass 1 adsorbing the reaction-involved substances 3 was placed in 1.33 mL of a 100 mM Tris buffer (pH 7.4) containing 20 mM ethylenediaminetetraacetic acid (EDTA), where the buffer was contained in a glass vessel. The glass vessel was closed up with a rubber stopper and was used as a reaction vessel. The porous glass 1 was irradiated with light from a simulated sunlight generator to promote a photochemical reaction. Gases were collected from the glass vessel using a syringe, and evolved hydrogen gas was detected and measured by gas chromatography. As a result, 6 nmol of hydrogen gas were detected after light irradiation for 4 hours.

### REFERENCE SIGNS LIST

1 porous glass
2 pore
3 reaction-involved substance

1. A photochemical reactor comprising:
porous glass having a lot of pores and being permeable to light; and
photosensitizer; electron carrier; and reduction reaction catalyst which are disposed in the pores.

2. The photochemical reactor according to claim 1, wherein the porous glass has an average pore diameter of 20 to 75 nm.

3. The photochemical reactor according to claim 1, wherein the electron carrier comprises one selected from the group consisting of methylviologen, cytochromes, methylene blue, and titanium oxides.

4. A method for producing the photochemical reactor according to claim 1, the method comprising:
step for preparing solution and the porous glass, the solution containing the photosensitizer, the electron carrier, and the catalyst; and
step for immersing the porous glass in the solution.

5. A method for performing a photochemical reaction, the method comprising:
preparing the photochemical reactor according to claim 1; and
in atmospheric air, supplying solution containing reactant material into the pores of the porous glass and applying the light to the porous glass.
